(19) United States
(12) Patent Application Publication
(10) Pub. No.: US 2021/0001017 A1
(43) Pub. Date: Jan. 7, 2021

(54) METHOD AND DEVICE FOR SURGERY

(71) Applicant: HERIOT EYECARE PTY. LTD., Melbourn, Victoria (AU)

(72) Inventor: Wilson J. Heriot, Melbourne, Victoria (AU)

(21) Appl. No.: 16/979,365

(22) PCT Filed: Mar. 8, 2019
(86) PCT No.: PCT/AU2019/000034

§ 371 Date: Sep. 9, 2020

(30) Foreign Application Priority Data
Mar. 9, 2018 (AU) 2018900775

(51) Int. Cl. A61L 31/14; A61F 9/007; A61F 2/12; A61L 31/04; A61L 31/06

(52) U.S. Cl. CPC A61L 31/14; A61F 9/00736; A61B 2017/00938; A61L 31/048; A61L 31/06; A61F 2/12

(57) ABSTRACT

The invention provides devices, methods and kits for surgery. More specifically, the invention provides a device for surgery comprising: a body comprising a handle; and one or hydrophobic or oleophilic extension or probe, wherein the device is adapted for removal of hydrophobic or oleophilic fluid. A method is also provided for removal of hydrophobic or oleophilic fluid, the method comprising: contacting the hydrophobic or oleophilic fluid with one or more hydrophobic or oleophilic extension or probe to thereby remove the hydrophobic or oleophilic fluid, whereby removal is by one or more of cohesive attraction, adhesion and capillary action.

### FIELD OF THE INVENTION

The present invention relates to a method and device for surgery. More particularly, this invention relates to a method and device for surgery using one or more extension or probe to assist in the removal of fluid.

### BACKGROUND TO THE INVENTION

Many surgical methods require the removal of hydrophobic or oleophilic fluid such as, silicone oil. Just one example is vitreous surgery, or vitrectomy, which is surgery to remove some, or all, of the vitreous from the eye. The vitreous, or vitreous humor, is the clear gel that fills the space between the lens and the retina of the eyeball of humans and other vertebrates. Anterior vitrectomy involves removing small portions of the vitreous from the front structures of the eye. Pars plana vitrectomy is a general term for operations accomplished in the deeper part of the eye, which remove some, or all, of the vitreous.

The vitreous that is removed may be replaced with a fluid such as, a saline solution, for example Balanced Salt Solution (BSS), or silicone oil, or with a gas.

An infusion line supplying either a fluid or gas is usually used to maintain constant eye pressure and volume. After removal of vitreous, the fluid in the cavity is frequently exchanged with a gas and less frequently with silicone oil or a derivative to keep the retina in place postoperatively. Any gas left in the eye will dissipate slowly and does not require any intervention. When silicone oil is used, a later operation is required to remove it.

The addition of silicone oil as a tamponade has become a standard technique and improves the prognosis of complex retinal detachment. Due to its high surface tension, the silicone oil does not enter the subretinal space. As the surface tension decreases, the silicone oil globule begins to emulsify. Because the silicone oil leads to long term complications, removal of silicone oil from the eye is recommended.

Conventionally, silicone oil is removed via one or more sclerotomy (incision into the sclera) while using one or more imaging modality, most commonly viewed through an operating microscope under direct visualisation for fundus control. A vacuum pump can then be used to suction off the silicone oil through an aspiration canula. Removal may also be passive, in which, after starting the cannula on fluid, one of the ports is kept open for passive egress of the silicone oil. This is followed by multiple fluid-air exchanges to ensure that the emulsified silicone oil globules trapped in the retro-iridial plane are flushed and removed. After the silicone oil is removed, the retina is inspected with a wide-angle visualization system for retinal pathology.

A hybrid technique has been reported in which the silicone oil is removed using a three-port, 20- and 23-gauge technique via a pars plana approach (RETINA PEARLS: Hybrid Technique for Silicone Oil Removal, Pearls for reducing complications. Manish Nagpal, MS, DO, FRCS(UK); Rituraj Videkar, MD; and Navneet Mehrotra, MD, Retina Today, March 2011; see: http://retinatoday.com/2011/03/hybrid-technique-for-silicone-oil-removal!). The infusion cannula and light pipe are 23 gauge; the oil removal port for active suction is 20 gauge in order to speed up the process of aspirating oil through a larger bore cannula. This hybrid technique is described as being less traumatic to the conjunctiva and allowing faster rehabilitation without compromising the surgeon's ability to tackle intraoperative events.

Another method is described as “Soup and Sandwich” and employs the standard three-port pars plana approach using a wide-angle viewing system (‘Soup & Sandwich’ Silicone Oil Removal, Skimming technique avoids consequences of other approaches. Rachel Trussart, MD, FRCSC, Mark S. Mandelcorn, MD, FRCSC, Efrem D. Mandelcorn, MD, FRCSC, and Robert G. Devenyi, MD, MBA, FRCSC, FACS; Retina Specialist, A publication by Review of Ophthalmology, Nov. 15, 2016; see: http://www.retina-specialist.com/article/soup-and-sandwich-silicone-oil-removal). The silicone oil is aspirated actively using a 23-gauge automated viscous extraction device (Alcon Constellation) while infusing BSS. To improve the removal of silicone oil from the vitreous cavity, an air-fluid exchange is conducted using a back-flush cannula. With the patient in the supine position, the air injected pushes back the silicone oil toward the posterior pole, sandwiching it between the air and the BSS.

To remove the oil overlay gathering on the interface, the tip of the back-flush cannula is placed at the level of the air-fluid interface where a thin film of silicone oil can be observed. Even when the air-fluid exchange is taken down to the optic nerve some oil remains on the fluid meniscus and probably some adheres to the retinal surface itself. The vitreous cavity is then partially infused with BSS which rinses the retinal surface of residual silicone oil. The BSS is re-infused cautiously at a low flow rate to avoid the stream from creating an iatrogenic retinal break. The remaining air acts as a barrier by precluding the residual oil from traveling anteriorly again and allows suspended silicon oil droplets to rise to the water/air interface. In a similar manner, multiple sequential air-fluid exchanges are completed until all the oil has been skimmed off.

Microdroplets persisting despite multiple fluid gas exchanges can collect in the anterior chamber superiorly as a hyperoleum and these can be associated with glaucoma, partially obscure vision in a “snowstorm” of very small droplets, or remain as large droplets seen by the patient when bending forward so that the oil floats up to the macula.

There remains a need for alternative techniques for the removal of the remaining of hydrophobic fluids. This can be silicone oil such as, the emulsified silicone oil, which may be present in micro droplets or as a film on the fluid surface, from the eye at the time of the surgery, fluid from a ruptured breast implant or silicone oil added during any surgical technique.

The reference to any prior art in this specification is not, and should not be taken as, an acknowledgement or any form of suggestion that the prior art forms part of the common general knowledge.

### Object of the Invention

It is a preferred object of the embodiments of the present invention to provide an apparatus that addresses or at least ameliorates one or more of the aforementioned problems of the prior art and/or provides a useful commercial alternative.

### SUMMARY OF THE INVENTION

Generally, embodiments of the present invention relate to methods and devices for surgery.

In a broad form, the invention relates to use of one or more extension or probe to assist in the removal of fluid.

In a first form, although it need not be the only or indeed the broadest form, the invention resides in a method for removal of hydrophobic or oleophilic fluid, the method comprising:

contacting the hydrophobic or oleophilic fluid with one or more hydrophobic or

oleophilic extension or probe to thereby remove the hydrophobic or oleophilic fluid.

In a first particular embodiment of the first form the removal is by one or more of cohesive attraction, adhesion and capillary action. The removal may be specific or substantially specific for the hydrophobic or oleophilic fluid.

According to the first particular embodiment of the first form, no extraneous or additional force or energy is applied. The removal is by intrinsic forces.

In a second particular embodiment of the first form, the method further includes the step of aspiration to assist in removal of the hydrophobic or oleophilic fluid.

According to the second particular embodiment of the first form, extraneous or additional force or energy in the form of aspiration is applied.

When the method is a method of eye surgery, the method may further include the step of creating an incision in the sclera, i.e. performing a sclerotomy.

When the method is a method for eye surgery, the method may further comprise filling of the eyeball with extraneous gas or fluid. The filling may be partial or complete. The extraneous fluid may be BSS.

In a second form, the invention resides in a device for surgery comprising:

a body comprising a handle; and

one or hydrophobic or oleophilic extension or probe.

The device for surgery of the second form may be for removal of hydrophobic or oleophilic fluid.

In a first particular embodiment of the second form the device removes the hydrophobic or oleophilic fluid by one or more of cohesive attraction, adhesion and capillary action. The removal may be specific or substantially specific for the hydrophobic or oleophilic fluid.

According to the first particular embodiment of the second form, the device does not apply any extraneous or additional force or energy.

In a second particular embodiment of the second form, the device applies aspiration to assist in removal of the hydrophobic or oleophilic fluid.

According to the second particular embodiment of the second form, extraneous or additional force or energy in the form of aspiration is applied.

In a third form, the invention resides in a method for manufacturing a device for surgery comprising:

disposing one or more one or hydrophobic or oleophilic extension or probe on a body comprising a handle.

In a fourth form, the invention resides in a kit for surgery comprising the device of the second form. The kit may further comprise an aspiration device and/or instructions for use. The aspiration device is any device that can apply an aspirating force. The aspiration device may be or may comprise the Alcon Constellation device, the Synergetics VersaVIT 2.0 Vitrectomy System, The D.O.R.C (Dutch Ophthalmic) EVA Phaco-Vitrectomy System, the Bausch+Lomb Stellaris PC or any other suitable commercially available device or one or more component thereof.

In one embodiment of any above form, the one or more of cohesive attraction, adhesion and capillary action is between the one or more hydrophobic or oleophilic extension or probe and the hydrophobic or oleophilic fluid.

In another embodiment of any above form, the one or more of cohesive attraction, adhesion and capillary action may be the result of non-covalent bonding. The non-covalent bonding may comprise one or more of electrostatic interactions, van der Waals forces, π-effects and the hydrophobic effect. The van der Waals forces may comprise London dispersion forces.

In yet another embodiment of any above form, the removal by one or more of cohesive attraction, adhesion and capillary action may further comprise one or more of wicking and mopping.

In one embodiment of any above form the one or more hydrophobic or oleophilic extension or probe comprises one or more of polypropylene (PP) and polyester (PE). The polyester may comprise Coolmax. The one or more hydrophobic or oleophilic extension or probe may be obtained from the School of Fashion & Textiles or Centre for Materials Innovation and Future Fashion (CMIFF) at RMIT University, Melbourne, Australia.

In one embodiment of any above form, the one or more hydrophobic or oleophilic extension or probe may comprise a large surface area. The one or more hydrophobic or oleophilic extension or probe may comprise one or more hydrophobic or oleophilic fibres; mesh; or open weave. The open-weave may be coral-like. The extension or probe may be like the brushes of a paintbrush.

The one or more extension or probe may be produced by melt extrusion and/or wet spinning. In a particular embodiment, the one or more extension or probe comprises polyester produced through melt spinning.

The one or more extension or probe may be of any suitable dimension, such as width, length and thickness.

In an embodiment, the one or more extension or probe may comprise a diameter from 50 micron to 700 microns.

In another embodiment, the one or more extension or probe may comprise a diameter from 1 micron to 50 microns, or from 5 micron to 40 microns, or from 10 micron to 25 microns.

In a particular embodiment, the one or more extension or probe may comprise a diameter of up to 50 microns. The one or more extension or probe may comprise a diameter of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 35; 40; 41; 42; 43; 44; 45; 46; 47; 48; 49; or 50 microns.

In a preferred embodiment of any of the above forms, the one or more hydrophobic or oleophilic fibres comprise one or more nanofibers and/or one or more microfibres. It will be appreciated that the fibres may be provided as one or more bundles.

In another embodiment of any of the above forms, the handle may be for gripping by a clinician or by a robot. The clinician may be an ophthalmologist or a surgeon.

In still another embodiment of any one of the above forms, the one or more hydrophobic or oleophilic extension or probe may be disposed to make contact with hydrophobic or oleophilic fluid in a body part. The body part may comprise an eye. The hydrophobic or oleophilic fluid may be in the form of a droplets or film. The droplets may be microdroplets. The film may be a fine film.

In yet another embodiment of any one of the above forms, the handle may be at a proximal end of the device.

In another embodiment of any one of the above forms, the one or more hydrophobic or oleophilic extension or probe may be at a distal end of the device.

In still another embodiment of any one of the above forms, the device may comprise a bore. The bore may allow passage of air for aspiration of fluid from the eye. The bore may comprise an internal bore. The fluid may comprise or consist of the hydrophobic or oleophilic fluid.

In another embodiment of any one of the above forms, the bore may comprise 5; 6; 7; 8; 9; 10; 11; 12; 13; 14; 15; 16; 17; 18; 19; 20; 21; 22; 23; 24; 25; 26; 27; 28; 29; 30; 35; 40 or greater than gauge diameter

In yet another embodiment of any one of the above forms, the method or device comprises a method or device for eye or vitreous surgery.

In still another embodiment the fluid may be removed from a body surface such as, the retina or another intraocular surface such as, a surface of an intraocular lens (IOL).

In yet another embodiment, the fluid may be removed from any part throughout the body; a tissue plane; or organ. The fluid removed may comprise fluid from a ruptured breast implant; oil under the conjunctiva; or fluid leaked into the orbit. The fluid removed may comprise silicone oil used in surgery, i.e. extraneous or extrinsic fluid.

In yet another embodiment of any one of the above forms, the method and device may comprise a flute needle.

In still another embodiment of any one of the above forms, the method and device may comprise a leak free connection. The leak free connection may comprise a Luer fitting, a Luer lock or a Luer taper. The device may further comprise an internal channel connecting a male Luer terminal to an exit port on the side of the device or of the handle. The exit port may be disposed in a depression. The exit port may be closed by a finger or robotic component to prevent flow of fluid or gas from the eye. Removal of the finger or robotic component allows egress of fluid along the needle and through the exit port.

According to any one of the above forms, an infusion of gas or fluid may be provided to the eye to maintain the intraocular pressure. The intraocular pressure may be maintained above atmospheric.

In still another embodiment of any one of the above forms, the method and device may comprise a collection chamber. The collection chamber may be in fluid communication with the one or more hydrophobic or oleophilic extension or probe.

The collection chamber may comprise a hydrophobic and/or oleophilic material. The hydrophobic and/or oleophilic material may comprise a mesh. The mesh may be porous. The mesh may comprise one or more of polypropylene (PP), polyethylene (PE), polyethylene terephthalate (PET); polyester; silicon; or mixtures thereof.

In still another embodiment of any one of the above forms, the method and device may comprise a mesh. The mesh may be disposed in the bore.

Further forms, aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1A, 1B, 1C and 1D illustrate the problems with the current technique to remove oil from the eye. FIG. 1A shows an oil-filled eye. FIG. 1B shows partial removal of the oil with BSS infusion posteriorly. FIG. 1C shows oil microdroplets in suspension after infusion agitation. FIG. 1D shows oil microdroplets on the surface an in the BSS.

FIG. 2 is a flowchart illustrating the steps according to one embodiment of a method of the invention.

FIG. 3 shows one embodiment of a device according to the invention.

FIGS. 4A; 4B; 4C; and 4D show, in sequence, the silicone oil slick (4A), the device according to one embodiment of the invention approaching (4B), touching (4C) and after showing the removal of silicone oil from the surface to the fibres (4D).

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

The present invention relates to a method and device for surgery. Embodiments of the present invention relate to a method and device for surgery using one or more extension or probe to assist in the removal of hydrophobic or oleophilic fluid.

While the invention will be described with reference to the removal of silicone oil, it is not so limited. For example, the invention may be used to remove fluid from a breast implant.

As used herein the terms “hydrophobic” means the absence or attraction or seemingly repelled from a mass of water and includes nonpolarity.

Herein the term “lipophilic” means the ability to dissolve in fats, oils, lipids, and non-polar solvents and includes non-polarity. This term, lipophilic, also encompasses, oleophilic which is used to mean having or relating to an affinity for non-polar compounds, oils or lipids.

As used herein the phrase “hydrophobic or lipophilic fluid” is used to mean one or both of “hydrophobic” and “lipophilic”. While the skilled person readily appreciates that lipophilic, hydrophobic and nonpolar are not synonymous, they describe the same tendency towards participation in the London dispersion force. The phrase “hydrophobic or lipophilic fluid” is then intended to mean having properties that allow it to effect removal of the fluid.

While the invention will be described with reference to the removal of silicon oil during vitreous surgery, it is not so limited. The extraneous silicone oil settles on the extraneous fluid, often BSS, in a density driven manner. The extraneous silicone oil may be present as either multiple microdroplets or as a film. There is some controversy in the field over whether the silicone oil microdroplets form by shear action or technical emulsification due to phospholipid (detergent-like) action. However, there is global acceptance that, regardless of their cause and physicochemical origin, the silicone oil micro droplets or film should be removed. If not, they can form a hyperoelium, glaucoma, retinal atrophy and optic nerve damage or a visual snow storm due to the opacities.

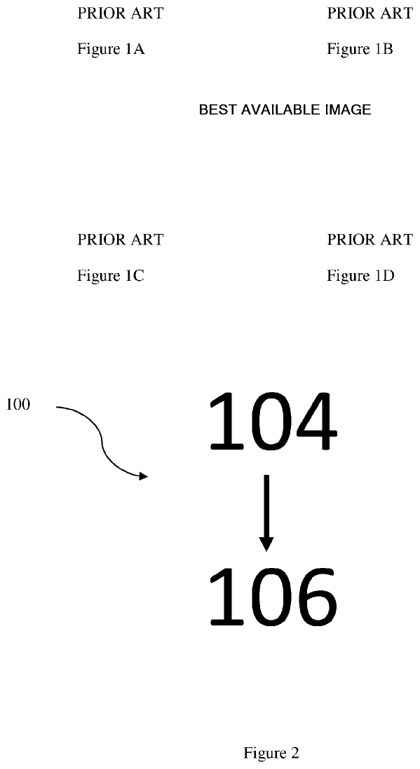

Through diligent research, the present inventor has sought an improved method and device for the removal of silicone oil. While the present invention has particular application to the eye, see FIG. 1A, it is not so limited. The inventor has found that when removing silicone oil from the eye, microdroplets or a film always remain (see FIGS. 1C and 1D). A fluid gas exchange can be conducted to remove the BSS fluid filled with microdroplets of oil. However, the problem remains that with a standard flute needle, a lot of the fluid is removed as well as the oil on the surface (see FIG. 1B) so that the fluid level drops. Even by trying to only aspirate the BSS, surface oil will remain on the meniscus of the residual fluid on the posterior retina and also probably covering the entire retinal surface. Turning on the BSS infusion again simply agitates the fluid, which can create an emulsion or dispersion—like making salad dressing. This results in the silicone oil droplets being resuspended in the turbulent fluid which again starts the same inefficient cycle.

The invention is at least partly predicated on the unexpected discovery that one or more hydrophobic or oleophilic extension or probe can be used to remove oil droplets or oil film floating on the surface of fluid with minimal aspiration of the fluid. In the vitreous surgery application, this advantageously, minimises the number of BSS refills that cause recurrent agitation.

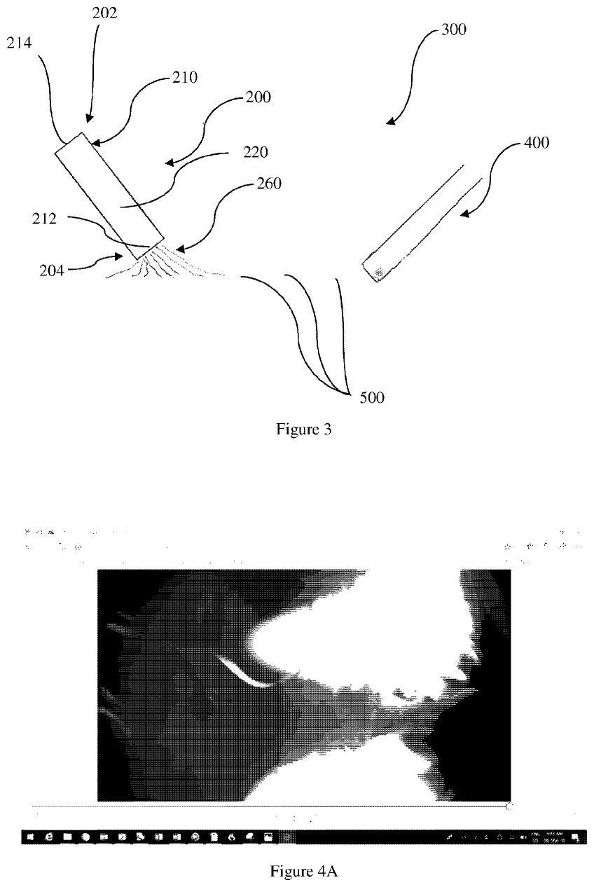

A flowchart showing one embodiment of a method 100 according to the invention is shown in FIG. 2. In step 104 hydrophobic or oleophilic fluid such as, silicone oil in the eye, is contacted with one or more hydrophobic or oleophilic extension or probe disposed on an aspiration device.

The contact between the one or more hydrophobic or oleophilic extension or probe and the hydrophobic or oleophilic fluid results from or by one or more of cohesive attraction, adhesion and capillary action. The contact and resulting cohesive attraction, adhesion and/or capillary action simplifies the removal. When the one or more hydrophobic or oleophilic extension or probe is removed or withdrawn, the silicone oil is also removed or withdrawn.

Advantageously, the removal or withdrawal is specific or substantially specific for the hydrophobic or oleophilic fluid.

The one or more of cohesive attraction, adhesion and capillary action may be the result of non-covalent bonding such as electrostatic interactions, van der Waals forces, π-effects and the hydrophobic effect. The van der Waals forces may comprise London dispersion forces.

As will be readily appreciated by a skilled person, this removal or withdrawal by one or more of cohesive attraction, adhesion and capillary action may further comprise one or more of wicking and mopping.

The contact and resulting cohesive attraction, adhesion and/or capillary action means that no extraneous or additional force or energy needs to be applied to achieve removal or withdrawal. The cohesive attraction, adhesion and/or capillary action are intrinsic forces.

As will be readily appreciated by a skilled person, the collection chamber, is in fluid communication with the one or more hydrophobic or oleophilic extension or probe such that when the one or more hydrophobic or oleophilic extension or probe is in contact with hydrophobic or oleophilic fluid, the fluid flows along the probe and is captured in the collection chamber. The capture may be by, or aided by, one or more hydrophobic and/or oleophilic material. The hydrophobic and/or oleophilic material may comprise a mesh. The capture of the fluid in the collection chamber, as will be appreciated by the skilled person, is advantageous as it reduces clogging of the one or more hydrophobic or oleophilic extension or probe and associated adverse effects on the flow of the fluid.

Method 100 is also shown to include the optional step of aspirating 106. The aspiration may be of the hydrophobic or oleophilic fluid and/or other extraneous fluid such as BSS.

The skilled person will readily appreciate when aspiration is used, extraneous or additional force or energy is required in addition to the cohesive attraction, adhesion and/or capillary action.

Method 100 may further include creating 102 an incision in the sclera, i.e. performing a sclerotomy.

The skilled person will readily appreciate that method 100 may further comprise providing an infusion of extraneous gas or extraneous fluid to the eye to maintain the intraocular pressure above atmospheric pressure. The filling may be partial or complete. The fluid may be BSS.

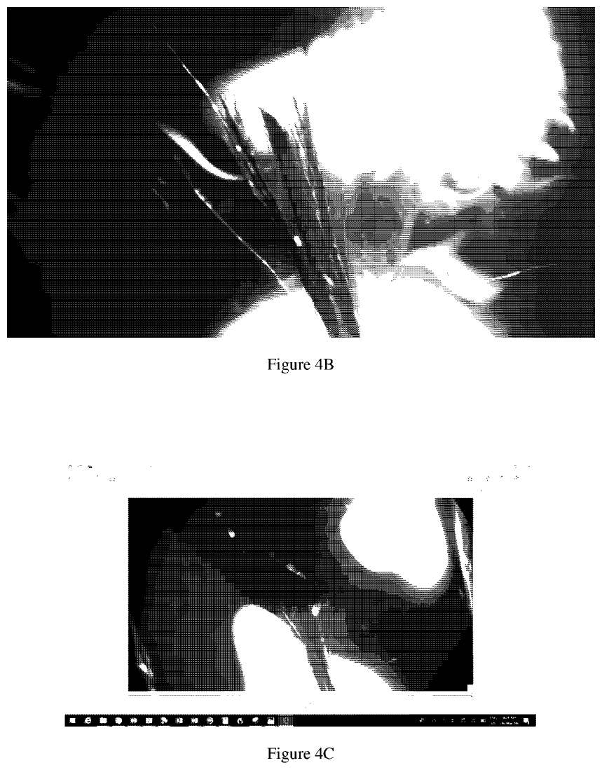

The invention also provides a device for surgery. One embodiment of a device 200 according to the invention is shown in FIG. 3. Device 200 comprises a body 210 comprising a handle 214, a collection chamber 213 (not shown), and one or hydrophobic or oleophilic extension or probe 260 in the form of fibres.

As will be readily appreciated by the skilled person, the one or more hydrophobic or oleophilic extension or probe 260 may be selected to have a large surface area. In addition to the one or more fibres shown in FIG. 3, the one or more extension or probe 260 may comprise one or more mesh; or an open weave. The open-weave may be coral-like. The extension or probe 260 may be like the brushes of a paintbrush.

From the teaching herein, a skilled person is readily able to select a suitable material for the one or more hydrophobic or oleophilic extension or probe 260.

Provision of handle 214 is advantageous for gripping by a clinician or by a robot. The clinician may be an ophthalmologist or a surgeon. Handle 214 is disposed at a proximal end of the device. In some embodiments, the collection chamber 213 is disposed intermediate the handle 214 and the proximal end of the device.

As shown in FIG. 3, the one or more hydrophobic or oleophilic extension or probe 260 is/are disposed to make contact with hydrophobic or oleophilic fluid. As shown in FIG. 3, the one or more hydrophobic or oloeophilic extension or probe 260 is/are disposed at a tip 212 a distal end 204 of device 200 (left of FIG. 3).

The one or more extension or probe 260 may be produced by melt extrusion and/or wet spinning. In the embodiment shown, the one or more extension or probe 260 comprises a plurality of fibres comprise of polyester produced through melt spinning.

From the teaching herein a skilled person is readily able to select suitable dimensions for the one or more extension or probe 260. For example, when provided as microfibers, the one or more extension or probe 260 may comprise a dimeter of 150 microns to 700 microns. The dimensions can be tailored by changing die and/or drawing method.

Device 200 comprises an internal bore 220 which allows passage of air for aspiration of fluid. The fluid may comprise or consist of the hydrophobic or oleophilic fluid 500. As will be appreciated from the above explanation of removal with intrinsic force along, in other embodiments device 200 is not required to have a bore or to provide aspiration.

From the teaching herein a skilled person is readily able to select a suitable bore. The bore may comprise 5; 6; 7; 8; 9; 10; 11; 12; 13; 14; 15; 16; 17; 18; 19; 20; 21; 22; 23; 24; 25; 26; 27; 28; 29; 30; 35; 40 or greater than gauge diameter.

From the teaching herein, the skilled person readily understands that the hydrophobic or oleophilic fluid may be removed from any body surface such as, any part throughout the body; a tissue plane; or organ. The fluid may be removed from the retina or another intraocular surface such as, a surface of an intraocular lens (IOL). The fluid removed may comprise fluid from a ruptured breast implant; oil under the conjunctiva; or fluid leaked into the orbit. The fluid removed may comprise silicone oil used in surgery, i.e. extraneous or extrinsic fluid.

Through provision of bore 220, device 200 may comprise a flute needle.

Device 200 is able to aspirate the surface silicone oil as the one or more hydrophobic or oleophilic extension or probe 260 is spread or spreads over the water meniscus into or on the oil droplets or film but do not enter the BSS. The conventional aspiration device 400, shown on the right in FIG. 3, a standard flute, is used to aspirate predominantly extrinsic BSS, or at least not the silicone oil, draining the eye and continuing the cycle. Any device that can apply an aspirating force is suitable as the conventional aspiration device 400.

Although not shown, device 200 may comprise a leak free connection 230. In one embodiment, the leak free connection 230 comprises a Luer fitting, also known as a Luer lock or a Luer taper (not shown). As will be readily appreciated by the skilled person, when device 200 comprises a Luer fitting, device 200 may further comprise a male Luer terminal connected via bore 220 to an exit port (not shown) on body 210 or handle 214. The exit port may be disposed in a depression (not shown). The exit port may be closed by a finger or robotic component to prevent flow of fluid or gas from the eye. Removal of the finger or robotic component allows egress of fluid along the needle and through the exit port.

The exit port may be disposed in the single channel bore 220 or may be in a secondary channel.

The method and device of the invention may be referred to as the SLIK WICK method and device.

Device 200 may be manufacturing by disposing the one or more one or hydrophobic or oleophilic extension or probe 260 on a body 210 comprising a handle 214.

The invention also provides a kit 300 for vitreous surgery comprising device 200 and optionally further comprising one or more of an aspiration device and instructions for use. In a preferred embodiment, the aspiration device may be or may comprise the Alcon Constellation device, the Synergetics VersaVIT 2.0 Vitrectomy System, any other suitable commercially available device or one or more component thereof.

The following non-limiting examples illustrate the invention. These examples should not be construed as limiting: the examples are included for the purposes of illustration only. The Examples will be understood to represent an exemplification of the invention.

### EXAMPLES

A prototype device 200 was manufactured using microfibers supplied by Dr Shadi Houshyar, Research Fellow, School of Fashion and Textiles, Royal Melbourne Institute of Technology. The prototype device 200 comprises a plurality of microfibres comprising polyester produced by melt spinning and comprising a diameter between 150 microns to 700 microns.

The device 200 comprises a body 210 in the form of a Luer lock (25 g needle) tube containing extension or probe 260, in the form of microfibers, that protrude from the tip 212 that is connected to an aspiration line 280 so that it acts like a “flute needle”.

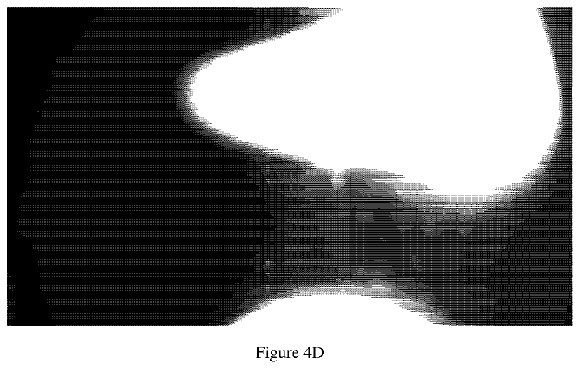

Initial trials showed excellent results with the one or more microfibres readily contacting and capturing silicone oil in an eye model with and without aspiration by a hand held syringe for enhanced ease of aspiration. FIGS. 4A to 4D show, in sequence, the silicone oil slick (4A), the device 200 approaching (4B), touching (4C) and after showing the removal of silicone oil from the surface to the fibres (4D). FIGS. 4A to 4D provide an impressive illustration of how effective the device and method of invention is.

The initial trials have shown that the use of microfibres provided a device resembling a very fine paintbrush or a woven meshwork of these fibres, where the oleophilic fibres optimise absorption of the oil because they stay on the BSS air/water meniscus and make contact with the oil preferentially to skimming the surface whilst minimising aspiration of the BSS.

The use of a collection chamber in fluid communication with the extension or probe 260 has the advantage of reducing clogging of the one or more hydrophobic or oleophilic extension or probe and associated adverse effects on the flow of the fluid.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

1. A device for surgery comprising:
a body comprising a handle; and
one or hydrophobic or oleophilic extension or probe,
wherein the device is adapted for removal of hydrophobic or oleophilic fluid.

2. The device of claim 1, wherein the device removes the hydrophobic or oleophilic fluid by one or more of cohesive attraction, adhesion and capillary action; and wherein the removal is at least substantially specific for the hydrophobic or oleophilic fluid.

3. The device of claim 1, wherein the device does not apply any extraneous or additional force or energy.

4. The device of claim 1, wherein the device applies aspiration to assist in removal of the hydrophobic or oleophilic fluid.

5. The device of claim 1, wherein the device comprises a bore to allow passage of air for aspiration of fluid from an eye, optionally, the bore comprising an internal bore.

6. The device of claim 5, wherein the bore comprises a diameter of 5; 6; 7; 8; 9; 10; 11; 12; 13; 14; 15; 16; 17; 18; 19; 20; 21; 22; 23; 24; 25; 26; 27; 28; 29; 30; 35; 40 or greater than, gauge.

7. The device of claim 1, for use in eye or vitreous surgery.

8. A method for removal of hydrophobic or oleophilic fluid, the method comprising:
contacting the hydrophobic or oleophilic fluid with one or more hydrophobic or oleophilic extension or probe to thereby remove the hydrophobic or oleophilic fluid, whereby removal is by one or more of cohesive attraction, adhesion and capillary action.

9. The method of claim 8, wherein the removal is at least substantially specific for the hydrophobic or oleophilic fluid.

10. The method of claim 8, wherein no extraneous or additional force or energy is applied to effect removal of the hydrophobic or oleophilic fluid.

11. The method of claim 8, wherein the method further includes the step of aspiration to assist in removal of the hydrophobic or oleophilic fluid.

12. The method of claim 8, wherein the method is a method of eye surgery and the method optionally includes the further step of creating an incision in the sclera, optionally further comprising filling of an eyeball with an extraneous gas or fluid, wherein the filling is partial or complete, and optionally, the extraneous fluid is BSS.

13. The device of claim 1, wherein the fluid is removed from a body surface, optionally a retina or another intraocular surface, or removed from a tissue plane; or organ.

14. The device of claim 13, wherein the fluid removed comprises fluid from a ruptured breast implant; oil under a conjunctiva; or fluid leaked into an orbit; optionally the fluid removed comprising silicone oil used in surgery.

15. The device of claim 13, further comprising:
(a) a leak free connection, wherein the leak free connection optionally comprises a Luer fitting, a Luer lock or a Luer taper; and/or
(b) a collection chamber, wherein the collection chamber is in fluid communication with the one or more hydrophobic or oleophilic extension or probe.

16. A kit for surgery comprising the device of claim 1, optionally further comprising an aspiration device and/or instructions for use.

17. The device of claim 2, wherein the one or more of cohesive attraction, adhesion and capillary action is a result of non-covalent bonding; optionally wherein the non-covalent bonding comprises one or more of electrostatic interactions, van der Waals forces, π-effects, hydrophobic effect and London dispersion forces, and optionally wherein the removal by one or more of cohesive attraction, adhesion and capillary action further comprises one or more of wicking and mopping.

18. The device of claim 2, wherein the one or more hydrophobic or oleophilic extension or probe comprises one or more of polypropylene (PP) and polyester (PE).

19. The device of claim 2, wherein the one or more hydrophobic or oleophilic extension or probe comprises a diameter from 50 microns to 700 microns, optionally 150 microns to 700 microns.

20. The device of claim 19, wherein the one or more hydrophobic or oleophilic extension or probe comprise a large surface area, optionally wherein the one or more hydrophobic or oleophilic extension or probe comprise one or more hydrophobic or oleophilic fibres; mesh; or open weave.
